(19) United States
(12) Patent Application Publication
(10) Pub. No.: US 2024/0169607 A1
(43) Pub. Date: May 23, 2024

(54) X-RAY IMAGING APPARATUS AND IMAGING PROCESS OF THE SAME

(71) Applicants: Woorien Co., Ltd., Gyeonggi-do (KR); VATECH EWOO Holdings Co., Ltd., Gyeonggi-do (KR)

(72) Inventors: Taehee HAN, Gyeonggi-do (KR); In CHOI, Gyeonggi-do (KR)

(73) Assignees: Woorien Co., Ltd., Gyeonggi-do (KR); VATECH EWOO Holdings Co., Ltd., Gyeonggi-do (KR)

(21) Appl. No.: 18/515,760

(22) Filed: Nov. 21, 2023

(30) Foreign Application Priority Data
Nov. 21, 2022 (KR) 10-2022-0156509

(51) Int. Cl. G06T 11/00; A61B 6/03; A61B 6/14; G01N 23/046

(52) U.S. Cl. CPC G06T 11/003; A61B 6/032; A61B 6/14; G01N 23/046; G06T 2207/10072; G06T 2207/10116

(57) ABSTRACT

An X-ray imaging apparatus and a method thereof are proposed. The apparatus includes an X-ray generator for emitting X-rays toward parts of an object to be imaged, an X-ray sensor for receiving the X-rays and obtaining projection image data for the parts of the object to be imaged, a driving part for moving the X-ray generator and the X-ray sensor along at least one partial section of the object to be imaged and causing the X-ray sensor to obtain a plurality of pieces of projection image data for the at least one partial section, and an image processor for obtaining a plurality of pieces of partial projection image data corresponding to partial areas of the X-ray sensor from the plurality of pieces of projection image data, and reconstructing tomographic images of the at least one partial section by using the plurality of pieces of partial projection image data.

### CROSS REFERENCE TO RELATED APPLICATION

The present application claims priority to Korean Patent Application No. 10-2022-0156509, filed Nov. 21, 2022, the entire contents of which are incorporated herein for all purposes by this reference.

### BACKGROUND OF THE INVENTION

### Field of the Invention

The present disclosure relates to an X-ray imaging apparatus and a method thereof and, more particularly, to an apparatus and method for providing X-ray tomographic images corresponding to various dental arch shapes.

### Description of the Related Art

X-ray panoramic images are a sort of X-ray tomographic images that are spread out and displayed along image layers close to a dental arch trajectory by way of shifting and adding a plurality of pieces of X-ray projection image data captured along a dental arch of a patient. X-ray panoramic images have been widely used in the field of dentistry for a long time, and are now recognized as essential standard images for diagnosis. Since lesions in the entire area of a set of teeth may be observed through images on one screen, the X-ray panoramic images are recognized as very useful during oral treatment, not only in dental clinics but also in animal hospitals.

However, in the case of humans, dental arches do not often deviate in shape from a standard dental arch significantly, but in the case of animals, dental arches are very diverse in their shapes. Even in the case of companion dogs, there are significant differences in the shapes of dental arches depending on the dog breeds. For now, the related art is in the process of proposing technological alternatives depending on the characteristics of animals on the basis of oral X-ray panoramic imaging technology that has been developed primarily for people as objects to be imaged, but there are many problems to be solved.

The present applicant is consistently researching and developing a technology for improving the quality of X-ray panoramic images captured from oral cavities of animals as well as humans in such a way of proposing an X-ray panoramic image acquisition technology that may overcome differences in tooth angles between upper and lower jaws of an animal in Korean Patent No. 10-2215249 (Feb. 5, 2021), and also proposing a panoramic imaging technology that may avoid physical interference from a quadrupedal animal while performing a panoramic imaging sequence and prevent artifacts from occurring in specific areas due to the characteristics of jaw skeletons of dogs in Korean Patent No. 10-2225773 (Mar. 4, 2021).

### DOCUMENTS OF RELATED ART

### Patent Documents

(Patent Document 1) Korean Patent No. 10-2215249 (Feb. 5, 2021)
(Patent Document 2) Korean Patent No. 10-2225773 (Mar. 4, 2021)

### SUMMARY OF THE INVENTION

An objective of the present disclosure is to provide an X-ray imaging apparatus and a method thereof capable of obtaining high-quality X-ray tomographic images corresponding to X-ray panoramic images with respect to an entire section through a single imaging sequence for various shapes of dental arches, e.g., even for a dental arch in a shape where a direction of a set of teeth is sharply bent for each section of the dental arch.

According to the present disclosure to solve the above-described problem, there is provided an X-ray imaging apparatus including: an X-ray generator for emitting X-rays toward parts of an object to be imaged; an X-ray sensor for receiving the X-rays and obtaining projection image data for the parts of the object to be imaged; a driving part for moving the X-ray generator and the X-ray sensor along at least one partial section of the object to be imaged and causing the X-ray sensor to obtain a plurality of pieces of projection image data for the at least one partial section; and an image processor for obtaining a plurality of pieces of partial projection image data corresponding to partial areas of the X-ray sensor from the plurality of pieces of projection image data, and reconstructing tomographic images of the at least one partial section by using the plurality of pieces of partial projection image data.

The partial areas may face toward a vertical direction of the X-ray sensor.

The object to be imaged may be a dental arch, the one partial section may include first and second sections at positions different from each other in the dental arch, the partial areas of the X-ray sensor for the first and second sections may be different from each other, and first and second tomographic images for the first and second sections may be first and second X-ray panoramic images having X-ray incidence angles different from each other.

According to the present disclosure, the X-ray imaging apparatus may further include a display, wherein the image processor may display the first and second panoramic images at positions different from each other on the display.

Here, the one partial section may include a first molar tooth section including molar teeth on a first side of the dental arch, a first canine tooth section including canine teeth on the first side, an anterior tooth section including anterior teeth, a second canine tooth section including canine teeth on a second side, and a second molar tooth section including molar teeth on the second side, and the image processor may display the first molar tooth section, first canine tooth section, anterior tooth section, second canine tooth section, and second molar tooth section on the display in order from the first side to the second side.

According to one aspect of the present disclosure, there is provided a method of realizing X-ray images, the method including: obtaining a plurality of pieces of projection image data for at least one partial section of an object to be imaged by using an X-ray generator for emitting X-rays toward parts of the object to be imaged and an X-ray sensor for receiving the X-rays transmitted through the parts of the object to be imaged to obtain the projection image data for the parts of the object to be imaged; and obtaining a plurality of pieces of partial projection image data corresponding to partial areas of the X-ray sensor from the plurality of pieces of projection image data; and reconstructing tomographic images for the at least one partial section by using the plurality of pieces of partial projection image data.

According to one aspect of the present disclosure, there is provided an X-ray imaging apparatus including: a storage for storing a plurality of pieces of projection image data for a plurality of projection images for at least one partial section of an object to be imaged, wherein a plurality of pieces of partial projection image data corresponding to partial areas of the projection images may be obtained from the plurality of pieces of projection image data, and tomographic images for the at least one partial section may be reconstructed by using the plurality of pieces of partial projection image data.

According to the present disclosure, there is provided an X-ray imaging apparatus and a method thereof capable of obtaining high-quality X-ray tomographic images corresponding to X-ray panoramic images with respect to an entire section through a single imaging sequence for various shapes of dental arches, e.g., even for a dental arch in a shape where a direction of a set of teeth is sharply bent for each section of the dental arch.

### BRIEF DESCRIPTION OF THE DRAWINGS

FIG. 1 is a view illustrating an outward form of an X-ray imaging apparatus according to an exemplary embodiment of the present disclosure.

FIG. 2 is a view illustrating capturing panoramic images of a companion dog by using the X-ray imaging apparatus according to an exemplary embodiment of the present disclosure.

FIG. 3 is a view schematically illustrating movement of an imaging part when panoramic images are captured by the apparatus according to the exemplary embodiment of FIG. 2.

FIG. 4 is a view illustrating incidence angle distribution for each sensor area of X-ray beams reaching an X-ray sensor of FIG. 3.

FIG. 5 is a view illustrating projection image data of a plurality of frames obtained from the X-ray sensor of FIG. 3.

FIG. 6 is a view illustrating a plurality of panoramic images provided with X-ray incidence angles different from each other and reconstructed by projection image data for each area of FIG. 5.

FIG. 7 is a view illustrating matching of a proper X-ray incidence angle (i.e., a sensor area) for each section depending on a shape of a dental arch to be imaged.

FIG. 8 is a view illustrating an example of displaying partial panoramic images for each section according to the example of FIG. 7.

FIG. 9 is a view illustrating a process of obtaining X-ray panoramic images according to the exemplary embodiment of the present disclosure.

### DETAILED DESCRIPTION OF THE INVENTION

Hereinafter, various exemplary embodiments of the present disclosure will be described with reference to the drawings. The technical idea of the present disclosure may be more clearly understood through the exemplary embodiments. The present disclosure is not limited to the exemplary embodiments described below. The same reference numerals indicate components of the same nature, and the description of components described with reference to the previous drawings may be omitted below.

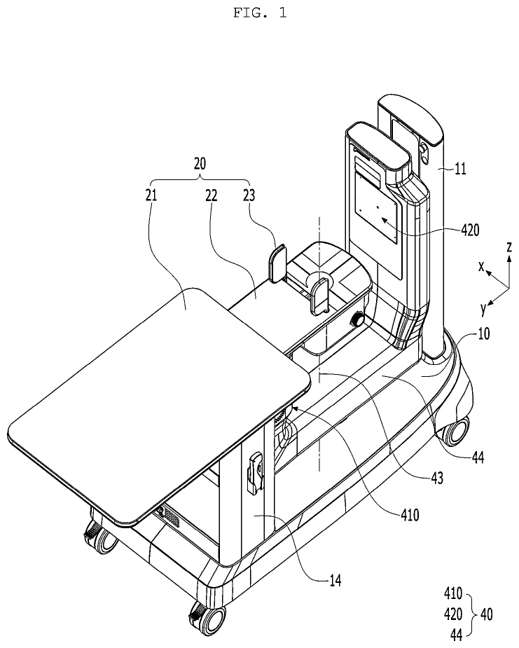

FIG. 1 illustrates an outward form of an X-ray imaging apparatus according to an exemplary embodiment of the present disclosure.

An X-ray imaging apparatus 100 according to the present exemplary embodiments is configured to include: a cart-type frame 10; a front support part 11 positioned in the front of the cart-type frame 10; a rear support part 14 positioned at the rear; a bed part 20 installed on the rear support part 14 to support an object to be imaged; and an imaging part 40 installed to be rotatable relative to the cart-type frame 10 between the rear support part 14 and the front support part 11. The imaging part 40 may be configured to include: a generator part 410; a sensor part 420; and a rotary arm 44 configured to connect the generator part 410 and sensor part 420 to each other and be rotatable relative to one rotation axis 43. In addition, although not shown, the X-ray imaging apparatus 100 according to the present exemplary embodiments includes: a driving part configured to enable an X-ray sensor to obtain a plurality of pieces of projection image data for at least one partial section by rotating an X-ray generator of the generator part 410 and an X-ray sensor of the sensor part 420 in opposing directions and moving the X-ray sensor along the at least one partial section of the object to be imaged.

The bed part 20 extends from a position where the rotation axis 43 passes to an outside of a rotation radius of the generator part 410 to support the object to be imaged. Along a longitudinal direction from the rear to the front, the bed part may be provided with the wide width part 21 having a relatively wide width and the narrow width part 22 having a relatively narrow width. The wide width part 21 is disposed on the rear support part 14 to support a body weight of an animal to be imaged, and the narrow width part 22 formed in the form of a cantilever extending forward is disposed to include a part intersecting the rotation axis 43.

When the longitudinal direction of the bed part 20 is defined as a y-axis direction and a direction perpendicular to the y-axis on a horizontal plane parallel to a floor is defined as an x-axis direction, the rotation axis 43 may be configured to move in the x-axis direction. The horizontal movement of the rotation axis 43 is not limited to linear movement in the x-axis direction described above. However, it is advantageous for the rotation axis 43 to move in the x-axis direction in order to provide an imaging sequence suitable for a shape of a dental arch of a companion dog V or the like to be imaged and to avoid collisions between the imaging part 40 and the front support part 11 or rear support part 14, which is a vertical structure of the cart-type frame 10.

In the bed part 20, the narrow width part 22 where a head part of an animal to be imaged is mainly positioned may be further provided with side support parts 23 for supporting both sides of the head part, and the side support parts 23 may be configured to allow position adjustment or transformation. For reference, in the drawing, the bed part 20 is shown as installed on the rear support part 14 of the cart-type frame 10, but may also be configured with another device separated from the cart-type frame 10.

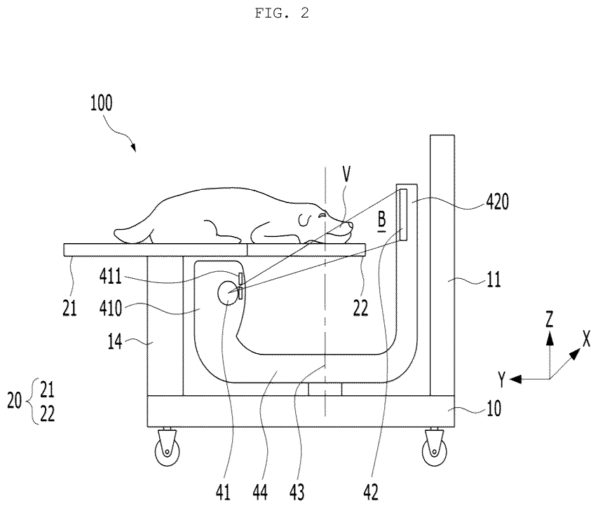

FIG. 2 illustrates capturing panoramic images of a companion dog by using the X-ray imaging apparatus according to an exemplary embodiment of the present disclosure.

The present drawing illustrates a configuration of the X-ray imaging apparatus 100 according to the present exemplary embodiments in an easy-to-understand manner through a view in which oral X-ray panoramic images of a companion dog V as an example of an object to be imaged is being captured. The generator part 410 is configured to include an X-ray generator 41 and emit cone beam-shaped X-ray beams B. The sensor part 420 is configured to include an X-ray sensor 42 for receiving the X-rays B transmitted through the object to be imaged.

In the above-described imaging part 40, the generator part 410 on which the X-ray generator 41 is installed has a lower height than the sensor part 420 on which the X-ray sensor 42 is installed. In the X-ray generator 41, X-ray beams B emitted through a collimator 411 penetrates an oral cavity part of the companion dog V to be imaged diagonally from bottom to top and reaches the X-ray sensor 42. Such a configuration of the bed part 20 and imaging part 40 provides advantageous effects in several aspects. First, a rotation trajectory of the generator part 410 may pass under the bed part 20, which is advantageous in terms of apparatus miniaturization. In addition, X-rays B may be emitted from the X-ray generator 41 to the X-ray sensor 42 at a proper inclination relative to a horizontal plane, whereby such a configuration also helps improve the quality of X-ray panoramic images of oral cavities of animals having sizes and shapes of the upper and lower jaws different from each other.

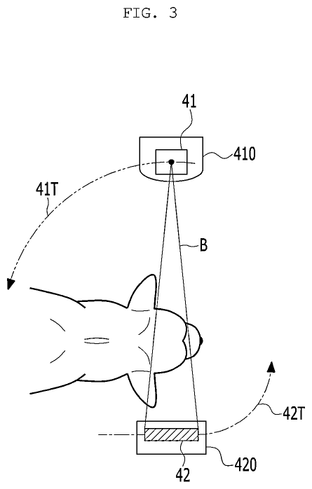

FIG. 3 schematically illustrates movement of the imaging part when panoramic images are captured by the apparatus according to the exemplary embodiment of FIG. 2.

The present drawing shows a part of a movement trace 41T of the X-ray generator 41 and a part of a movement trajectory 42T of the X-ray sensor 42 on an xy plane of FIG. 2 when an X-ray panoramic imaging sequence is performed along at least one partial section of a dental arch. At least during the panoramic imaging sequence, a position of the X-ray generator 41 is relatively fixed within the generator part 410 and a position of the X-ray sensor 42 is relatively fixed within the sensor part 420. Since there is no change in the relative positions of the generator part 410 and sensor part 420 in the imaging part, the X-ray generator 41 and X-ray sensor 42 as the entire imaging part rotate in opposing directions without change in their positions relative to each other. As described above, the rotation axis of the imaging part may be moved.

The X-ray sensor 42 has a predetermined width in a peripheral direction of the movement trace 42T. The predetermined width may have a width that is larger than an eight mm width of a sensor that has been mainly applied to conventional panoramic imaging apparatuses and is smaller than a width (e.g., about 10 cm) of a conventional CT imaging sensor. The generator part 410 emits X-ray beams B corresponding to the width of the X-ray sensor 42, and as a result, the X-rays reach various parts at incidence angles different from each other along the width direction of the X-ray sensor 42.

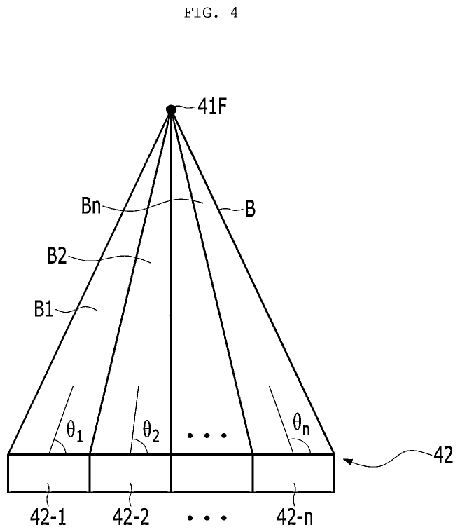

FIG. 4 illustrates incidence angle distribution for each sensor area of X-ray beams reaching an X-ray sensor of FIG. 3.

For the sake of easy understanding, when the X-ray sensor 42 is divided into n areas (where n is a natural number greater than 1) having an elongated shape toward a vertical direction, the X-ray sensor 42 may be divided into n sensor areas from a first sensor area 42-1 to an n-th sensor area 42-n including a second sensor area 42-2 stating from the left side of the drawing on the basis of positions in the width direction. Here, the n sensor areas are not areas physically divided but are virtually-divided sensor areas associated with a subsequent panoramic image reconstruction process. The number n of the sensor areas may also be changed as required.

As shown, among X-ray beams B emitted from an X-ray focus 41F, X-rays passing through a first beam path B1 are emitted to a first sensor area 42-1 and have an average incidence angle of θ₁. Among the X-ray beams B, X-rays passing through a second beam path B2 are emitted to a second sensor area 42-2 and have an average incidence angle of θ₂. Among the X-ray beams B, X-rays passing through an n-th beam path Bₙ are emitted to an n-th sensor area 42-n and have an average incidence angle of θₙ. These first to n-th sensor areas 42-1 to 42-n respectively correspond to the vertically long strip-shaped areas (in a z-axis direction of FIG. 1) on a sensing surface of the X-ray sensor 42.

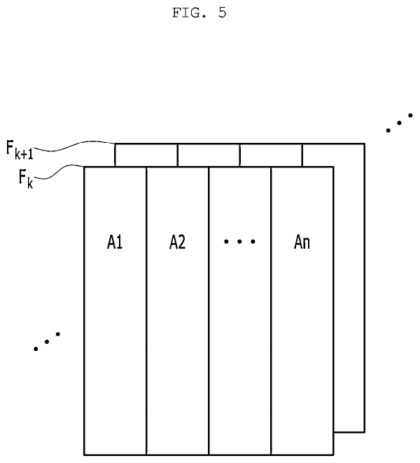

FIG. 5 illustrates projection image data of a plurality of frames obtained from the X-ray sensor of FIG. 3.

During a panoramic imaging sequence, a plurality of frames of X-ray projection image data F₁, . . . , Fₖ, Fₖ+1, . . . is obtained from the X-ray sensor 42 in a form of images of consecutive frames. For example, referring to an arbitrary k-th frame Fₖ of the projection image data, as described above, the frame Fₖ is configured to include a first part A1 obtained from the first sensor area 42-1 and a second part A2 obtained from the second sensor area 42-2, and in the same manner, is configured to include an n-th part obtained from the n-th sensor area 42-n. Similarly, in addition to a (k+1)-th frame Fₖ+1, each frame is configured to include a first part A1 to an n-th part An. As described above, the first part A1 to n-th part An are formed with projection images, i.e., transmission images, obtained by X-rays having respective average incidence angles of θ₁ to θₙ.

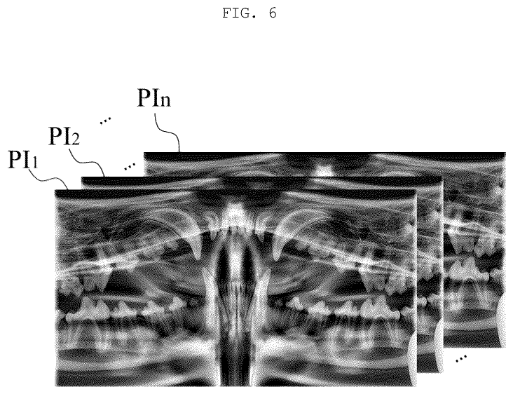

FIG. 6 illustrates a plurality of panoramic images provided with X-ray incidence angles different from each other and reconstructed by projection image data for each area of FIG. 5.

The X-ray imaging apparatus according to the present disclosure uses the plurality of frames of X-ray projection image data F₁, . . . , Fₖ, Fₖ₊₁, . . . previously obtained, but may reconstruct a plurality of panoramic images PI₁, PI₂, . . . , and PIₙ having X-ray incidence angles different from each other as many as the number of parts (same as the number of sensor areas) by reconstructing the panoramic images for each of the first part A1 to n-th part An.

To this end, in the plurality of frames of X-ray projection image data F₁, . . . , Fₖ, Fₖ₊₁, . . . , an image processor of the X-ray imaging apparatus according to the present disclosure may first divide each frame into first to n-th parts and reconstruct n panoramic images in a way of shifting and adding a first part, . . . , and an n-th part among themselves in the projection image data of each frame.

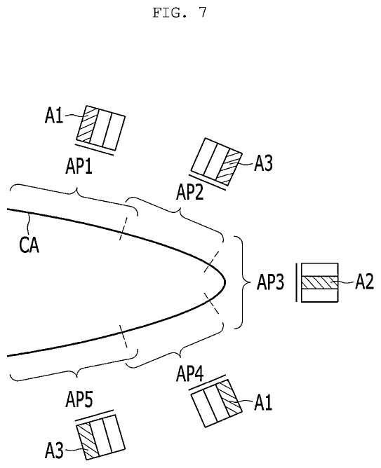

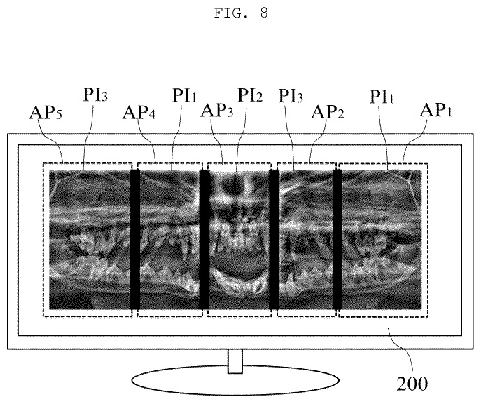

FIG. 7 illustrates matching of a proper X-ray incidence angle (i.e., a sensor area) for each section depending on a shape of a dental arch to be imaged. FIG. 8 illustrates an example of displaying partial panoramic images for each section according to the example of FIG. 7.

A dental arch of an animal, e.g., a dental arch of a companion dog, is narrower in width from side to side and longer from front to back compared to the shape of a dental arch of a human, and there is a section where a direction of canine teeth is sharply bent between an anterior teeth part and a canine teeth part. In addition, the sizes and shapes of dental arches vary greatly depending on dog breeds. In a case of capturing oral panoramic images of various companion dogs by using a standardized panoramic imaging sequence as in the past, the quality of the panoramic images becomes uneven because incidence directions of X-rays are not proper relative to directions of the teeth in some parts of each dental arch.

Accordingly, the X-ray imaging apparatus according to the present disclosure may divide a dental arch CA into several dental arch sections AP1 to AP5 in consideration of directions of teeth, etc., and match and provide a panoramic image reconstructed by image areas each having a most proper incidence angle for each dental arch section AP1, . . . , and AP5.

The present exemplary embodiment corresponds to a case in which the X-ray sensor is divided into three virtual sensor areas having X-ray incidence angles different from each other and three panoramic images are reconstructed by using partial projection image data A1, A2, and A3 corresponding to the respective sensor areas. As shown in the present drawing, the dental arch CA to be imaged is divided into a first molar tooth section AP1, a first canine tooth section AP2, an anterior tooth section AP3, a second canine tooth section AP4, and a second molar tooth section AP5. Partial panoramic images reconstructed by each piece of partial projection image data having proper incidence angles may be corresponded to the respective sections.

For example, panoramic images may be displayed on a viewer screen 200 of the apparatus by a method including: respectively matching corresponding section parts of the panoramic images PI₁ reconstructed by using the first part A1 projection image data with the first molar tooth section AP1 and second canine tooth section AP4; respectively matching corresponding section parts of the panoramic images PI₃ reconstructed by using the third part A3 projection image data with the first molar tooth section AP2 and second canine tooth section AP5; and matching a corresponding section part of the panoramic image PI₂ reconstructed by using the second part A2 projection image data with the anterior tooth section AP3. Through this way, images of even quality across all the sections of the dental arch may be provided.

The dividing of a dental arch into sections and matching of a proper incidence angle for each section may be determined in advance depending on the size or breed of an animal to be imaged. In this case, the X-ray imaging apparatus may be configured to pre-store information on the dental arch section division and section-specific incidence angle matching in a storage by type of object to be imaged, and display partial panoramic images (corresponding to the entire dental arch to be imaged) which are a group of X-ray tomographic images having section division and incidence angle matching different from each other on one screen depending on a choice of a person viewing the panoramic images through the viewer.

Meanwhile, the dental arch section division and the section-specific proper incidence angle matching may also be derived automatically by analyzing the plurality of panoramic images which are obtained in the method described with reference to FIG. 6, i.e., which are reconstructed from the projection image data having incidence angles different from each other, in the X-ray imaging apparatus according to one aspect of the present disclosure. In this case, matching an optimal partial panoramic image may be performed for each dental arch section by analyzing complexity for each area, strength of features, and the like of the panoramic images.

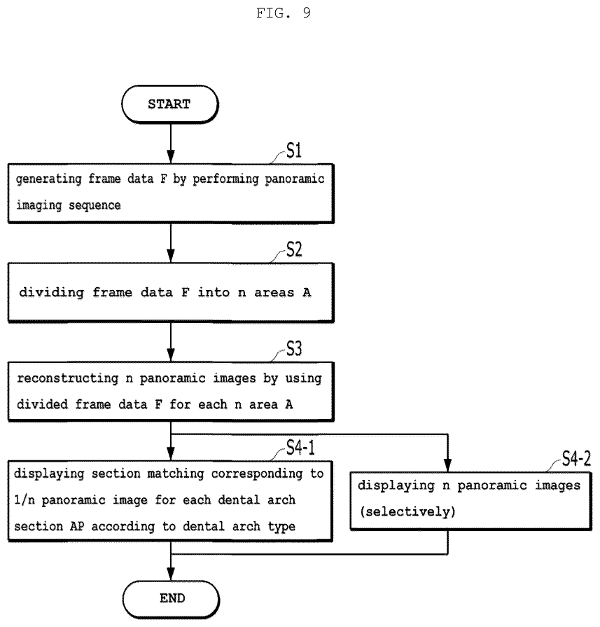

FIG. 9 illustrates a process of obtaining X-ray panoramic images according to the exemplary embodiments of the present disclosure.

In a method of obtaining X-ray panoramic images according to the exemplary embodiment of the present disclosure, first, in step S1, a panoramic imaging sequence is performed to obtain a plurality of frames of X-ray projection image data F. In step S2, the X-ray projection image data F in units of frame is divided into n pieces of partial projection image data A having X-ray incidence angles different from each other, and in step S3, a first to n-th panoramic images PI₁ to PIₙ are reconstructed by using the plurality of frames of X-ray projection image data for each of the first part A1 to n-th part An.

In step S4-1, the X-ray imaging apparatus may previously store information on panoramic image matching according to the dental arch section division and incidence angles, wherein a dental arch section AP is divided into first to m-th dental arch sections AP1 to APm depending on a type of dental arch to be imaged before a most proper one (1/n) panoramic image among first to n-th panoramic images PI₁ to PIₙ is matched for each divided dental arch section AP, and in this case, a proper panoramic image PIₓ (where x is a natural number from 1 to n) matched for each divided dental arch section AP may be displayed on a viewer screen of the X-ray imaging apparatus.

Meanwhile, in step S4-2, in a case where the information on the panoramic image matching according to the dental arch section division and incidence angles as described above is not secured in advance, the first to n-th panoramic images PI₁ to PIₙ reconstructed previously in step S3 may be displayed all or selectively on the viewer screen. In this case, the method may also be configured to divide the dental arch section according to determination of a person viewing panoramic images through the viewer and match corresponding parts of any one of proper X-ray tomographic images, i.e., the first to n-th panoramic images PI₁ to PIₙ, to respective sections (AP1 to APm, where m is a natural number greater than 1), thereby displaying the images on the screen.

In the above, the number n of panoramic images PI and the number m of dental arch sections AP according to the incidence angles may be the same natural number, but are not limited thereto. In addition, these numbers may also vary depending on an object to be imaged.

What is claimed is:

1. An X-ray imaging apparatus comprising:
an X-ray generator for emitting X-rays toward parts of an object to be imaged;
an X-ray sensor for receiving the X-rays and obtaining projection image data for the parts of the object to be imaged;
a driving part for moving the X-ray generator and the X-ray sensor along at least one partial section of the object to be imaged and causing the X-ray sensor to obtain a plurality of pieces of projection image data for the at least one partial section; and
an image processor for obtaining a plurality of pieces of partial projection image data corresponding to partial areas of the X-ray sensor from the plurality of pieces of projection image data, and reconstructing tomographic images of the at least one partial section by using the plurality of pieces of partial projection image data.

2. The X-ray imaging apparatus of claim 1, wherein the partial areas face toward a vertical direction of the X-ray sensor.

3. The X-ray imaging apparatus of claim 1, wherein the object to be imaged is a dental arch, the one partial section comprises first and second sections at positions different from each other in the dental arch, the partial areas of the X-ray sensor for the first and second sections are different from each other, and first and second tomographic images for the first and second sections are first and second X-ray panoramic images having X-ray incidence angles different from each other.

4. The X-ray imaging apparatus of claim 3, further comprising:
a display,
wherein the image processor displays the first and second panoramic images at positions different from each other on the display.

5. The X-ray imaging apparatus of claim 4, wherein the one partial section comprises a first molar tooth section comprising molar teeth on a first side of the dental arch, a first canine tooth section comprising canine teeth on the first side, an anterior tooth section comprising anterior teeth, a second canine tooth section comprising canine teeth on a second side, and a second molar tooth section comprising molar teeth on the second side, and the image processor displays the first molar tooth section, first canine tooth section, anterior tooth section, second canine tooth section, and second molar tooth section on the display in order from the first side to the second side.

6. A method of realizing X-ray images, the method comprising:
obtaining a plurality of pieces of projection image data for at least one partial section of an object to be imaged by using an X-ray generator for emitting X-rays toward parts of the object to be imaged and an X-ray sensor for receiving the X-rays transmitted through the parts of the object to be imaged to obtain the projection image data for the parts of the object to be imaged; and
obtaining a plurality of pieces of partial projection image data corresponding to partial areas of the X-ray sensor from the plurality of pieces of projection image data; and
reconstructing tomographic images for the at least one partial section by using the plurality of pieces of partial projection image data.

7. An X-ray imaging apparatus comprising:
a storage for storing a plurality of pieces of projection image data for a plurality of projection images for at least one partial section of an object to be imaged,
wherein a plurality of pieces of partial projection image data corresponding to partial areas of the projection images is obtained from the plurality of pieces of projection image data, and tomographic images for the at least one partial section are reconstructed by using the plurality of pieces of partial projection image data.
